(19) United States
(12) Patent Application Publication
(10) Pub. No.: US 2024/0058496 A1
(43) Pub. Date: Feb. 22, 2024

(54) CHEMICAL INJECTION AND METERING MODULE FOR STERILIZATION SYSTEM

(71) Applicant: American Sterilizer Company, Mentor, OH (US)

(72) Inventors: Fabio Di Bartolomei, Ardea (IT); Salvatore Gambuzza, Pomezia (IT); Fabio Nazzarri, Rome (IT); Lawrence J. Formosa, Robbinsdale, MN (US); Judson A. Herrig, Elko New Market, MN (US); Martin J. Kaib, Minneapolis, MN (US); Maruti Sinha, Plymouth, MN (US)

(21) Appl. No.: 18/259,293

(22) PCT Filed: Dec. 22, 2021
(86) PCT No.: PCT/US21/64813

§ 371 Date: Jun. 26, 2023

Related U.S. Application Data
(60) Provisional application No. 63/199,523, filed on Jan. 5, 2021.

(51) Int. Cl. A61L 2/26; A61L 2/24; A61L 2/18

(52) U.S. Cl. CPC A61L 2/26; A61L 2/24; A61L 2/18; A61L 2202/15; A61L 2202/14; A61L 2202/24

(57) ABSTRACT

The present disclosure discusses a sterilization system having a dosing module with a dosing cylinder to allow for metered flow of a predetermined amount of sterilant fluid into the sterilization system. The dosing module can include a dosing cylinder for measuring the predetermined amount of sterilant fluid, a pump for moving the sterilant fluid, and a controller configured to initiate driving of the sterilant fluid through the dosing module and into the sterilization system. A method of providing a predetermined amount of sterilant fluid to the sterilization system can include controlling the flow of sterilant fluid into the dosing cylinder, measuring the amount of sterilant fluid in the dosing cylinder to a desired dose amount, and controlling flow of the dose into the sterilization system.

### CLAIM OF PRIORITY

This application claims priority to and the benefit of U.S. Provisional application with Ser. No. 63/199,523, filed Jan. 5, 2021, entitled CHEMICAL INJECTION AND METERING MODULE FOR STERILIZATION SYSTEM, which is herein incorporated by reference in its entirety.

### BACKGROUND

Sterilization and disinfection systems, for example those systems used to sterilize medical equipment and devices, often are validated prior to use. Validation can, for example, ensure that the sterilization or disinfection system is functioning properly and killing or disabling a sufficient number of microorganisms to sterilize the equipment being treated. In some cases, validation can include confirming the amount or type of chemical composition inside a sterilization chamber or disinfection environment.

In a sterilization or disinfection device, cycles can generally contain a number of phases. For example, many sterilization or disinfection cycles can include a conditioning phase, such as where a vacuum is created in the system, an injection phase where a sterilant or disinfectant is inserted into the system, a sterilization or disinfection phase where the products are sterilized or disinfected by the sterilant or disinfectant, and a ventilation phase where the sterilant or disinfectant is vented out of the system. Due to these various phases, the number of microorganisms and the amount of sterilant or disinfectant in the system can significantly vary over the course of the system cycle.

In a sterilization procedure, a given amount of chemical sterilant can be used. Generally, the chemical sterilant is measured before insertion into the sterilization system. The amount of chemical sterilant can correlate to the particular instrument or items being sterilized, and the anticipated microorganisms thereon.

### SUMMARY OF THE DISCLOSURE

Disclosed is a dosing system and associated methods for measurement of chemical components being injected into a sterilization system. The dosing system allows for continuous, pulsed flow of the chemical components into a dosing cylinder, which can continuously measure the volume of chemical components received. Once a requisite dose is collected, the system can inject the appropriate volume of chemical components into the sterilization system.

In a sterilization system, a particular amount or concentration of chemical sterilant can be desired. Leveraging a precise amount of chemical sterilant desired can allow for more efficient and precise sterilization of instruments in the sterilization system. Measuring of chemical sterilant can be time consuming and cannot be easily repeatable, additionally measurement of chemical sterilant manually can require close human contact and potential accidents with chemical sterilant materials which may be corrosive.

The dosing system discussed herein can allow for contactless, repeatable dosing and measuring of chemical sterilant for a sterilization system. The system can also help minimize air gaps or bubbles that may occur in the chemical sterilant line during manual dosing. The dosing system and associated methods discussed herein can allow for improved accuracy in chemical sterilant dose delivery to a sterilization system.

In an example, a system for measuring chemical components can include a dosing cylinder configured to receive and measure a fluid containing one or more chemical components, the dosing cylinder including a fluid sensor, a pump operably coupled to the dosing cylinder, the pump configured to drive the fluid to the dosing cylinder, and a controller in communication with the dosing cylinder and the pump. The controller can be configured to: generate a signal to initiate driving of the fluid into the dosing cylinder, receive a signal from the fluid sensor in response to the fluid reaching a predetermined volume in the dosing cylinder, generate a signal to stop pumping of the fluid into the dosing cylinder, and dispersing the fluid from the dosing cylinder to a sterilization system.

In an example, a sterilization system can include a sterilization module, a dosing cylinder coupled to the sterilization module, the dosing cylinder for receiving and measuring a fluid containing one or more chemical components, the dosing cylinder including a fluid sensor, a pump operably coupled to the dosing cylinder, the pump for driving the fluid to the dosing cylinder, and controller in communication with the dosing cylinder and the pump. The controller can include a processor and a memory with instructions which, when executed, cause the processor to provide commands that: initiate driving of the fluid into the dosing cylinder, identify a condition, based on data received from the fluid sensor, the condition indicating that the fluid has reached a predetermined volume in the dosing cylinder, stop pumping of the fluid into the dosing cylinder in response to the condition, and initiate driving of the fluid of the predetermined volume from the dosing cylinder to the sterilization module.

In an example, a method can include controlling flow of a sterilant fluid into a dosing cylinder, measuring an amount of the sterilant fluid in the dosing cylinder, and stopping driving of the sterilant fluid into the dosing cylinder when a threshold amount is reached.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic diagram of a sterilization system in an example.

FIG. 2 is a schematic diagram of a dosing system for use with a sterilization system in an example.

FIGS. 3A-3B are perspective views of a dosing cylinder for use with a dosing system in an example.

FIG. 4A-4C are schematic diagrams of a dosing system having one pump for use with a sterilization system in a first example.

FIG. 5 is a schematic diagram of a dosing system having one pump for use with a sterilization system in a second example.

FIG. 6 is a block diagram depicting a method of dosing a sterilization system in an example.

### DETAILED DESCRIPTION

The present disclosure describes, among other things, a chemical sterilant dosing system for use with a sterilization system. The chemical sterilant dosing system can allow for continuous measurement and dosing of an injection volume of the chemical sterilant used in the sterilization system. The dosing system can include a separate dosing chamber, such as a cylinder, for receiving and measuring the incoming chemical sterilant, and subsequently administering the chemical sterilant in the appropriate dose to the sterilization system. The dosing chamber can be in communication with a controller for automated control of the measurement and injection of the chemical sterilant, such as continuously or for a desired amount of time prior to running a sterilization cycle on the sterilization system.

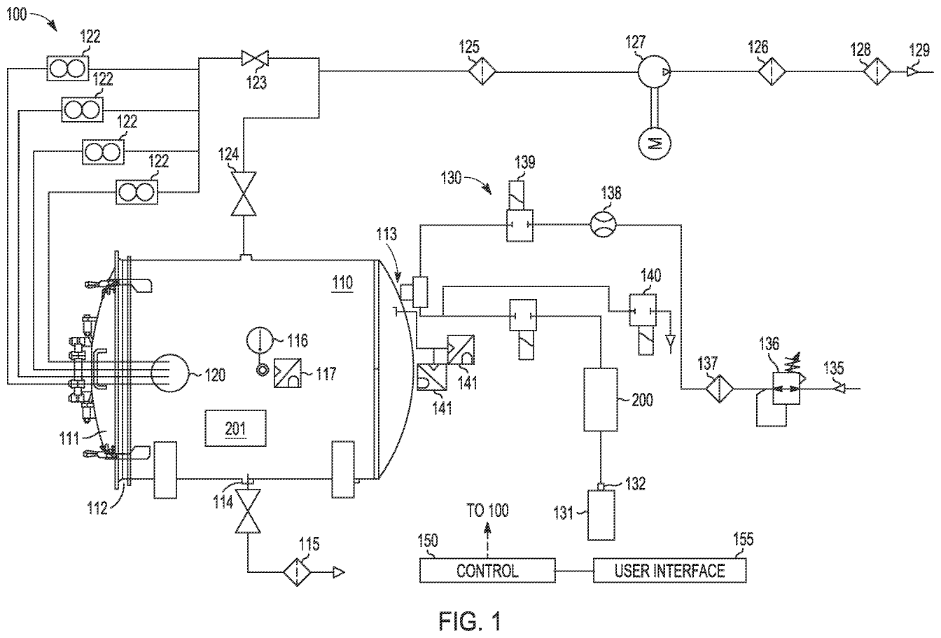

The dosing system can include, for example, a dosing cylinder with valves for inlet and outlet of the chemical sterilant, a sensor, such as an ultrasonic sensor, for detection of the amount or volume of chemical sterilant entering the dosing cylinder, and a dispensing pump, such as a micro-liquid or other appropriate fluid pump for administering doses of the chemical sterilant. The dosing system can be used in conjunction with a sterilization system, such as the example system shown in FIG. 1.

FIG. 1 is a schematic diagram of an example sterilization system 100 in which a dosing module 200 can be used. In other examples, a disinfection system can be used. The example sterilization system 100 can include, for example, sterilization chamber 110 with door 111, seal 112, atomizer inlet 113, vent 114, air filter 115, temperature sensor 116, and relative humidity sensor 117; vacuum hookup connection 120 with gas flow meters 122, vacuum control valve 123, chamber vacuum control valve 124, vacuum pumps 127, filters 125, 126, 128, and muffler 129; atomizer assembly 130 with sterilant 131, chemical inlet 132, air inlet 135, pressure regulator 136, air filter 137, air flow control 138, air valve 139, atomizer purge 140, and pressure transducers 141; in addition to controller 150 with user interface 155 and dosing module 200.

The sterilization chamber 110 can include, for example, a chamber for insertion of medical equipment or other items to be sterilized. The sterilization chamber 110 can be of any appropriate size for the items to be sterilized. For example, in some examples, the sterilization chamber 110 can be about 300 to about 500 L. In some examples, the sterilization chamber 110 can be about 2,000 to about 4,000 L. The sterilization chamber 110 can be accessed by a user by, for example, the door 111. The door 111 can be, for example, on one side of the sterilization chamber 110, and the door 111 can be large enough to allow for insertion of the items for sterilization. The door 111 can be, for example, airtight so that a vacuum can be applied inside the sterilization chamber 110 for sterilization of the medical equipment or other items. In some examples, the door 111 can be shut with a seal 112 in or around the door 111. In some cases, such as with a disinfection system, a disinfection environment can be used instead of a sterilization chamber.

The sterilization chamber 110 can have, in or on one or more sides, an atomizer inlet 113 and a vent 114. The atomizer inlet 113 can allow for influx of a chemical composition, such as one or more sterilant or disinfectant, into the sterilization chamber 110 to interact with and sterilize or disinfect the medical equipment or other items. The sterilant or disinfectant can be, for example, atomized, vaporized, or in other gaseous form, as desired, for sterilization.

The vent 114 in the sterilization chamber 110 can allow for venting of the sterilization chamber as desired. Venting of the environment inside the sterilization chamber can be performed, for example, to maintain a particular pressure inside the chamber, or to alter the pressure inside the chamber over time. An air filter 115, such as a HEPA air filter, can, for example, be in fluid communication with the vent 114, to allow gas or air to pass out of the sterilization chamber 110 and be filtered for chemicals, as the gas leaves the sterilization chamber 110.

The sterilization chamber 110 can also include, for example, a number of sensors, such as a temperature sensor 116, a relative humidity sensor 117, combinations thereof, or other sensors such as pressure, optical, flow rate sensors, or combinations thereof. The sensors 116, 117, or other additional sensors, can, for example, be in communication with a controller or processor, such as controller 150, to allow manipulation of the sterilization process in the sterilization chamber 110 based on parameters sensed, such as temperature or humidity.

The vacuum hookup connection 120 can connect the sterilization chamber 110 to a vacuum source. The vacuum hookup connection 120 can include, for example, the gas flow meters 122, the vacuum control valve 123, the chamber vacuum control valve 124, the vacuum filters 125, 126, 128 the vacuum pumps 127, and the muffler 129.

The vacuum hookup connection 120 can allow for an airtight connection between the sterilization chamber 110 and the vacuum system (e.g., components 120 to 129). The vacuum system can, for example, include one or more gas flow meters 122 to monitor the flow of gas in and out of the sterilization chamber 110 before, during, and after a sterilization cycle.

The valves 123, 124, can allow for specific control of the flow rate of gas in the vacuum system, so as to create a specific pressure inside the sterilization chamber 110. The vacuum filters 125, 126, 128, also along the vacuum lines, can allow for cleaning of air being used in the vacuum system. The filters can include, for example, polypropylene filters, sodium bicarbonate filters, potassium permanganate filters, and other types of filters appropriate for a vacuum sterilization system.

The vacuum pumps 127 can, for example, create a vacuum along the vacuum system so as to induce a vacuum in the sterilization chamber 110. The muffler 129 can, for example, minimize noise created by the vacuum system.

The atomizer assembly 130 can include, for example, a sterilant 131, a chemical inlet 132, an air inlet 135, a pressure regulator 136, an air filter 137, an air flow control 138, an air valve 139, an atomizer purge 140, and one or more pressure transducers 141. The dosing cylinder 200 can be used in conjunction with the atomizer assembly 130 to provide sterilant to the system 100.

In system 100, a sterilant is atomized for the sterilization process inside the sterilization chamber 110. In some sterilization systems, a sterilant is vaporized, or supplied as a liquid. In some sterilization systems, the sterilant is atomized or vaporized in a separate chamber, and then pumped or run into the sterilization chamber. The dosing module 200 can be used, for example, in combination with any of these types of sterilization systems.

In the example sterilization system 100 shown in FIG. 1, the sterilant 131 is stored as fluid in bottles. In some examples, other containers, with various shapes or storage methods, could be used. The sterilant 131 is connected to the chemical inlet 132 into the atomizer assembly 130, and the sterilant can be moved, for example, by the dosing cylinder 200.

At the air inlet 135 of the atomizer assembly 130, air (or other gas) can enter the atomizer assembly 130 for use in atomizing the sterilant. The pressure regulators 136 can monitor the pressure of the air in the atomizer assembly 130 to allow proper atomization of the sterilant. One or more air filters 137, air flow controls 138, and air valves 139, can be used in manipulating the flow of air and sterilant to induce atomization of the sterilant in the sterilization chamber 110. The atomizer purge 140 can allow for venting or dumping of excess air or sterilant depending on pressure feedback. The pressure transducers 141 can, for example, monitor the pressure of sterilant as it is atomized.

In system 100, the controller 150 can be in communication with the vacuum system, atomizer assembly 130, and the sterilization chamber 110. The controller 150 can, for example, include a processor and memory that executes instructions to receive and process sensed data from the various sensors in the system, and direct changes in the valves or air lines within the system. The controller 150 can, for example, manipulate the pressure in the sterilization chamber created by the vacuum system, and the amount and rate of atomized sterilant entering the system.

The controller 150 can be, for example, connected to a user interface 155, so as to communicate this information to the user. The user interface can, for example, include a screen, or one or more user-actuated buttons or triggers to allow to user to read information and alter the sterilization process as desired.

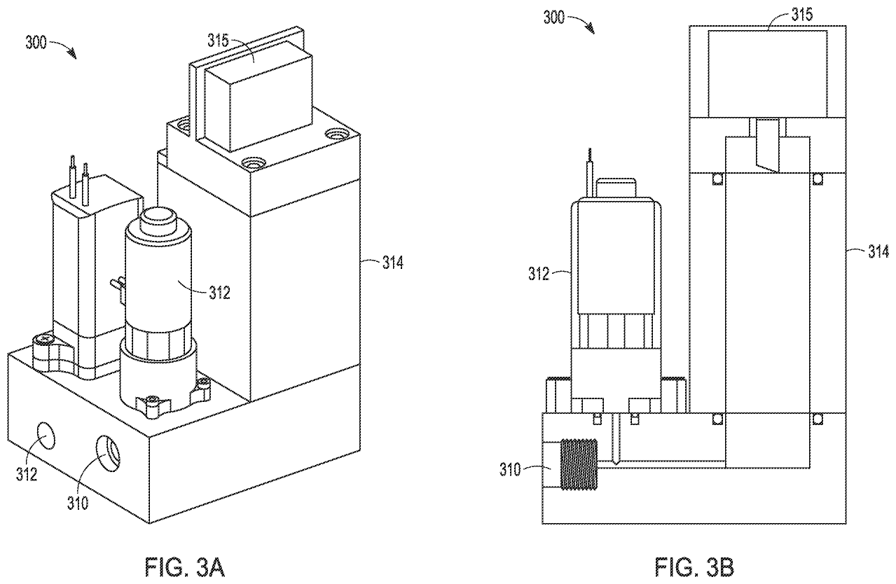

Load to be sterilized 201 can be used inside the sterilization chamber 110 of the system 100 to verify the sterilization processes of the system 100. The load to be sterilized 201 can be placed or located physically inside the sterilization chamber 110, either loose or mounted to the inside of the sterilization chamber 110. The load to be sterilized 201 can carry in it one or more indicators, such as biological indicators or chemical indicators, that can react with the atomized sterilant when a sterilization cycle is run on the system 100. The dosing module 200 and the load to be sterilized are discussed in more detail with references to FIGS. 3A-3B, 4A-4C, and 5 below.

The sterilization system 100 discussed herein is one example of a sterilizing or disinfecting system in which a sampling assembly, such as the load to be sterilized 201, can be used. A dosing module can be used in sterilization system 100 for provision of sterilant. In some examples, a different types of disinfection or sterilization system can be used.

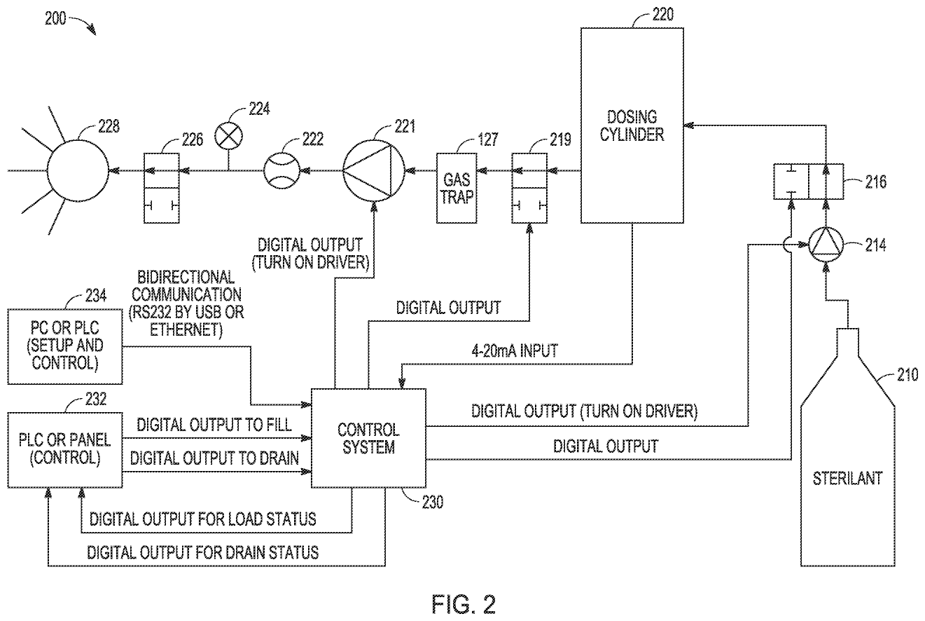

FIG. 2 is a schematic diagram of a dosing module 200 for use with a sterilization system. The dosing module 200 can function as a chemical injection and metering module, such as for use with a sterilization system. The components of the dosing module 200 can replace or supplement the components of the atomizer assembly 130 discussed above. The dosing module 200 can include a sterilant container 210, an inlet 212, a pump 214, a valve 216, a dosing cylinder 220, a valve 219, gas trap 127, a second pump 221, a flow meter 222, a pressure sensor 224, a valve 226, and an outlet 228. The dosing module 200 can be connected to an atomizer, such as the atomizer assembly 130 discussed with reference to sterilization system 100. The dosing module can be in communication with a controller 230, a computing unit 232, and a panel control 234.

The dosing module 200 can be used to administer measured and metered doses of a chemical sterilant to a sterilization system (such as system 100). In the dosing module 200, the chemical sterilant, stored in the sterilant container 210, is drawn up into the system towards the dosing cylinder 220 through the inlet 212 by the pump 214, through the first valve 216. In the dosing cylinder 220, the amount of chemical sterilant is measured. When an appropriate amount of chemical sterilant for a dose is determined, the second pump 221 can draw the appropriate dose of the chemical sterilant out of the dosing cylinder 220 towards the atomizer assembly 130. The flow meter 222 and the pressure sensor 224 can be in line with the dose of chemical sterilant for monitoring the dose being pumped towards the sterilization system 100. The controller 230, the computing unit 232, and the panel control 234, can be in communication with and control the action of the components along the line between the sterilant container 210 and the atomizer assembly 130.

The dosing module 200 can allow for an accuracy of final injection volume of about 1% to about 2%, within the tolerance of about 5% for many sterilization systems. The dosing module 200 can allow for dosing of amount down to about 0.30 microliters. If used with an atomizer, the dosing module 200 can allow for atomization at as low as about 3 psi, or in an about 5 to about 50 micron droplet size. The dosing module 200 can be run, for example, with parameters such as about a 0.5 mm orifice, about 10 psi air pressure, and a flow rate of about 2 mL per minute.

Specifically, the sterilant container 210 can be an appropriate material for holding a chemical sterilant such as a corrosive acid. Examples of chemical sterilant can include peracetic acid, acetic acid, hydrogen peroxide, or other appropriate chemicals. The sterilant container 210 can be fluidly connected to the dosing module 200 to allow for flow of sterilant from the sterilant container 210 toward the dosing cylinder 220. The sterilant container 210 can be coupled to the inlet 212 and the first pump 214.

The inlet 212 can include a valve 216 for selective pumping of the chemical sterilant towards the dosing cylinder 220. The liquid dispenser pump 214 can be a chemical pump used for drawing the chemical sterilant into the dosing module 200. The pump 214 can be, for example, a micro dispensing pump, such as for dispensing small amount of the chemical sterilant, for example about 5 microliters to about 200 microliters. The pump 214 can operate at a pressure of about 0.10 to about 0.35 bar.

The dosing cylinder 220 can be, for example, a continuous system for receiving, measuring, and dispensing incoming chemical sterilant. The dosing cylinder can be a chamber of a different shape or size, and in some cases can be a cylinder. The dosing cylinder 220 can include a sensor for detecting the amount of chemical sterilant in the dosing cylinder 220. In some cases, this can be an ultrasonic sensor, such as described with reference to FIGS. 3A-3B below. The sensor in the dosing cylinder 220 can detect the amount of chemical sterilant in the dosing cylinder, and can be configured to detect when a particular dose of chemical sterilant has been collected sufficient to pump the dose towards the sterilization system.

The second pump 221 can be coupled to the dosing cylinder to pump one or more doses of the chemical sterilant towards the sterilization system 100 when the dosing cylinder 220 detects an appropriate dose of the chemical sterilant is collected. The second pump can be coupled to the dosing cylinder 220, such as through the third valve 226. The second pump can, for example, include a diaphragm, such as a PTFE (polytetrafluoroethylene) diaphragm (or other appropriate material) for pumping the chemical sterilant dose towards the sterilization system 100 from the dosing cylinder 220. The second pump 221 can, for example, pump at a rate of about 0.20 to about 20 mL per minute. The second pump 221 can operate at a pressure of about 0.30 to about 6.0 bar. In some cases, the system can include a gas trap 127 between the valve 219 and the second pump 221. The gas trap 127 can act as a de-bubbler to remove air or gas bubbles before reaching the second pump 221.

The flow meter 222 and the pressure sensor 224 can be downstream of the second pump 221 and be used to monitor properties of the dose of chemical sterilant moving towards the sterilization system 100. The flow meter 222 can detect the flow of the sterilant dose, while the pressure sensor can detect the pressure in the line delivering the sterilant dose. The flow meter 222 can be, for example, a meter for detecting flow in the range of about 0 to about 4 mL per minute, with an outer diameter of about ⅛″. For example, the pressure sensor 224 can be configured to operate at a pressure of about 0 to about 70 psi.

Once flowing past the flow meter 222, and the pressure sensor 224, the chemical sterilant dose can reach the atomizer assembly 131 and be atomized for use in the sterilization system 100. In some cases, a liquid dispenser, a gaseous dispenser, or other type of dispenser can be used instead of an atomizer.

The controller 230 can be configured to regulate the measurement, metering and dosing of the chemical sterilant in the dosing module 200. The controller 230 can include various forms of circuitry, controllers, or other appropriate components. This can be implemented by circuitry that receives signals, provides digital outputs, receives digital outputs, or similar. The controller 230 can be in communication with several of the components of the dosing module 200, such as the dosing cylinder 220. The controller 230 can collect information, such as when a specified dose amount is collected in the dosing module 200, and initiate movement of that dose towards the sterilization system.

For example, the controller 230 can generate a signal to drive a sterilant fluid towards the dosing cylinder. In this case, the controller 230 can produce a signal o communicate with the appropriate valves, such as first valve 216, and the first pump 214, to allow open the valves and initiate the pump, such as to allow flow of the sterilant fluid into the dosing cylinder 220. The controller 230 can be in communication with the sensor in the dosing cylinder 220. The sensor can, for example, detect a height, volume, or other amount of sterilant fluid filling the dosing cylinder 220. In some cases, where a height of fluid is detected within the dosing cylinder 220, the controller 230 can be configured to convert this measurement signal into a volume amount. In some cases, the controller 230 can identify a condition, such as the sensor indicated a desired amount of sterilant has been reached in the dosing cylinder 220.

The controller 230 can allow for continuous or pulsatile flow of the sterilant fluid into the dosing cylinder 220 until the sensor in the dosing cylinder 220 detects a desired (e.g., a threshold or a predetermined volume associated with one dose of sterilant for the sterilization system) amount of sterilant, such as a desired dose. In this case, the sensor in the dosing cylinder 220 can send a signal to the controller 220 to indicate flow should stop.

When the controller 230 receives a signal that filling of the dosing cylinder 220 should halt, the controller 230 can generate a signal to stop the first pump 214 and close the first valve 216. At this time, the controller 230 can also generate a signal to disperse the measured dose of sterilant fluid from the dosing cylinder 220 towards the sterilization system. This signal can indicate, for example, that the valves 219 and 226 should open to allow fluid flow, and that the second pump 221 should initiate pumping of the sterilant fluid.

The computing unit 232 can be a personal computer (PC) or programmable logic controller (PLC) configured to perform set-up and control of the controller 230, and may include or be coupled to a user interface device for conveying information to the user and for initiating a sterilization sequence. The controller 230 may have a bi-directional wire communication link (e.g., a serial RS232, USB, ethernet, or other) with the computing unit 232. In other examples, wireless communication links can be used. The computing unit 232 can be in communication with the controller 230. Similarly, the panel control 234 can be a PLC or other user interface in communication with the controller 230 for initiating or halting a sterilization sequence. The PLC or panel control 234 can, for example, communicate with the controller 230 to initiate filling of the dosing cylinder 220, or draining of the dosing cylinder 220 towards to the sterilization system.

With the use of the computing unit 232, the PLC 234, and the controller 230, a user can initiate filling of the dosing cylinder 220 with a sterilant, through opening of the appropriate valves and initiation of the pump. The user can receive an indication that the dosing cylinder 220 has reached a desired (e.g., threshold) amount of sterilant for a dose based on data from the sensor, such as an ultrasonic sensor. At this time, the user can initiate a draining mode of the dosing module, where the valves allowing flow of new sterilant are closed, the first pump is halted. The second pump can be initiated and downstream valves opened to allow draining of the sterilant dosing from the dosing cylinder 220 towards a sterilization system. In some cases, this process can be automated by communication between the controller 230 and the other components of the module 200.

FIGS. 3A-3B are perspective views of a dosing cylinder 300 for use with a dosing system, such as system 200. The dosing cylinder 300 can be an ultrasonic or other measuring cylinder configured to meter our specific doses of a sterilant. The dosing cylinder 300 can be a nozzle delivery system, such as for receiving and dispersing a liquid sterilant. The dosing cylinder 300 can, for example, have operating parameters such as an operating temperature of about −90 to about 400° C., and a working pressure of about vacuum to 100 bar, depending on the dosing system in which the cylinder 300 is used. In some cases, the dosing cylinder 300 can have a resolution of less than about 0.1 mm when measuring doses. The dosing cylinder can be electrically coupled to other components in the dosing module 200, and in some cases can be an explosion-protected device.

FIG. 3A show a perspective view of the dosing cylinder 300, while FIG. 3B shows a side view of the dosing cylinder 300. The dosing cylinder 300 can include a sterilant inlet 310, a pump 312, a dosing chamber 314, and a sterilant outlet 316. The sterilant inlet 310 can receive an incoming sterilant, such as from a reservoir. The pump 312 can move the sterilant into the dosing chamber 314. The pump 312 can be, for example, a micro-pump, such as described above. The dosing chamber 314 can have a volume of about 0 to about 25 mL.

The pump 312 can move sterilant into the dosing chamber 314, where the amount of sterilant can be monitored, such as by an ultrasonic sensor 315. For example, an ultrasonic level sensor can be used inside the dosing chamber 314 to monitor the amount of sterilant present. An ultrasonic level sensor 315 can, for example, emit acoustic energy at a frequency too high for humans to hear. The sound can be reflected back to the sensor. Based on the time of reflection, the ultrasonic sensor can determine whether a fluid is present at a given level within the dosing cylinder 300.

The ultrasonic sensor 315 can, for example, use echo propagation time measurement at a frequency range of about 350 to about 400 kHz. The ultrasonic sensor can, in some cases, have an operating range of about 150 mm, a maximum range of about 250 mm, and a blind zone of about 20 mm. The ultrasonic sensors can have a resolution (sampling range) of about 0.056 mm+0.056 mm. In some cases, the ultrasonic sensor can have a reproducibility of about +0.15%, or about +1% accuracy. In some cases, other types of sensors can be used. When a desired dose is reached, the sterilant dose can be moved out the outlet 316 towards a sterilization system, such as through a nozzle.

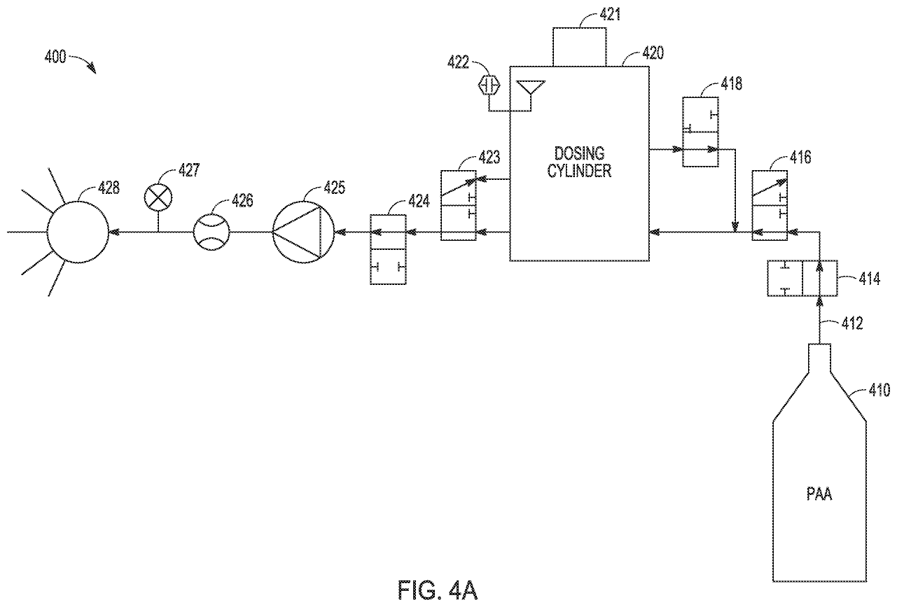

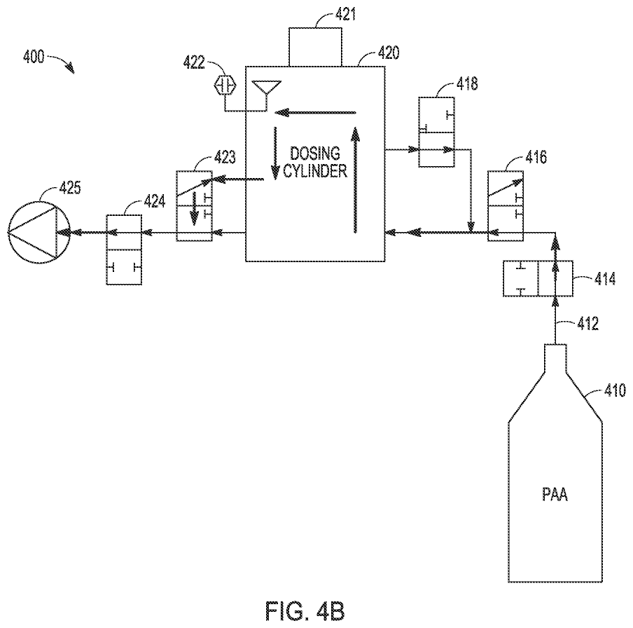

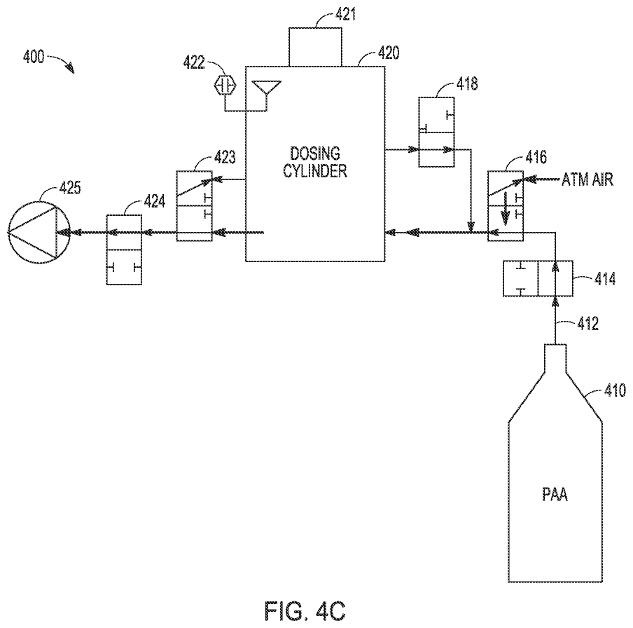

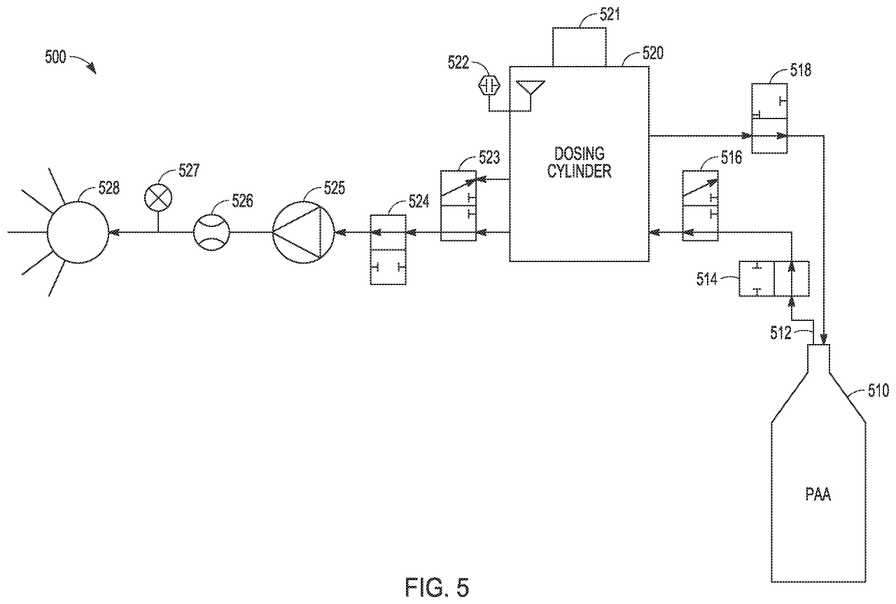

FIGS. 4A-4C and 5 depict variations on a dosing module using a single pump. FIG. 4A are schematic diagrams of a dosing module 400 having one pump 425 for use with a sterilization system. The dosing module 400 can be similar to dosing module 200 discussed above, and can have similar components. The dosing module 400 can include a sterilant container 410, an inlet 412, valves 414, 416, and 418, a dosing cylinder 420 with ultrasonic level sensor 421 and overflow level switch 422, valves 423, 424, a pump 425, a flow meter 426, a pressure sensor 427, and outlet 428 to a sterilization system. The dosing module 400 outlet 428 can be connected to an atomizer, such as the atomizer assembly 130 discussed with reference to sterilization system 100. The dosing module can be in communication with a controller and a user interface (not shown).

The dosing module 400 can work in a two-step function as shown in FIGS. 4B and 4C, where the arrows indicate sterilant flow within the module 400. In the first step, depicted in FIG. 4B, the dosing cylinder 420 can be filled. The first two valves 414 and 416 can be energized to allow flow of the sterilant from the sterilant container 410 toward the dosing cylinder 420. The first valve 414 on the inlet 412 can, for example, be a two-way valve. The second valve 416 can be a three-way valve. During this step, the valve 423 can be de-energized to prevent flow of sterilant out of the dosing cylinder 420. The single pump 425 can be turned on to promote flow of the sterilant.

In FIG. 4B, the sterilant can flow from the sterilant container 410 and fill the dosing cylinder 420 from the bottom to the top, until the ultrasonic level sensor 421 indicates that a desired level of sterilant has been reached in the dosing cylinder 420. In case of overflowing, the overflow level switch 422 can energize the overflow valve 418 to allow release of a portion of sterilant back towards the sterilant container 410.

In FIG. 4C, the second step can include dispensing from the dosing cylinder 420. In FIG. 4C, the valves 414 and 416 can be de-energized to prevent flow of the sterilant from the sterilant container 410. The valves 423 and 424 downstream of the dosing cylinder can be energized to allow for flow of the sterilant in a metered dose out towards a sterilization system. The single pump 425 can be turned on to promote flow of the sterilant. In this step, the pump 425 dispenses the chemical sterilant from the dosing cylinder 420 through the valves 423, 424, such as through a sprayer in the outlet 428. Air can be sucked into the dosing cylinder 420 through the valve 416 until the ultrasonic level sensor 421 reads zero.

FIG. 5 is a schematic diagram of a dosing module having one pump for use with a sterilization system in a second example. The dosing module 500 can be similar to dosing module 400 discussed above, and function in a similar two step manner. The dosing module 500 can include a sterilant container 510, an inlet 512, valves 514, 516, and 518, a dosing cylinder 520 with ultrasonic level sensor 521 and overflow level switch 522, valves 523, 524, a pump 525, a flow meter 526, a pressure sensor 527, and outlet 528 to a sterilization system. The dosing module 500 outlet 528 can be connected to an atomizer, such as the atomizer assembly 130 discussed with reference to sterilization system 100. The dosing module 500 can be run in a similar fashion to the steps described with reference to FIGS. 4A-4C above. In a single pump configuration, such as the dosing modules shown in FIGS. 4A-4C and 5, a single pump can allow for more efficient driving of sterilant towards the sterilization system while dosing that sterilant.

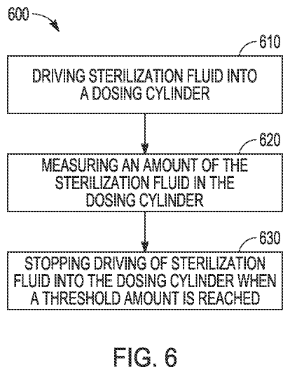

FIG. 6 is a block diagram depicting a method 600 of dosing a sterilant provided to a sterilization system. Method 600 can begin with driving a sterilant fluid into a dosing cylinder (block 610). For example, driving a sterilant fluid can include running a pump to promote movement of a sterilant from a sterilant source to a dosing cylinder, such as across a dosing valve, and moving the sterilant in a forward direction through the system. In this step, drain valves can be closed to prevent loss of sterilant while the dosing cylinder is being filled.

While the sterilant is being run into the dosing cylinder, the amount of sterilant fluid in the dosing cylinder can be measured. (Block 620). A sensor integrated with the dosing cylinder, such as an ultrasonic sensor, can be used to monitor the amount of sterilant present in the dosing cylinder.

When the desired amount (e.g., a threshold amount) of sterilant is detected in the dosing cylinder, the dosing valve can be closed to stop driving the sterilant into the dosing cylinder. (Block 630). In this case, the drain valve can be opened and the pump can be reversed to drain the cylinder towards a sterilization system with the appropriate dose of sterilant. In some cases, a vacuum can be used to draw the sterilant out. In some cases, a second, separate pump can be used to pump the sterilant out of the dosing cylinder and into a sterilization system.

In some cases, controlling flow of the sterilant fluid can include pumping the fluid in a pulsatile manner into the dosing cylinder. Pumping the fluid in a pulsatile manner can include pumping a plurality of pulses of the sterilant fluid, where each of the pulses has a volume of about 5 μL to about 200 μL. For example, the fluid flow can be moved from the sterilant container to the dosing cylinder continuously when the valves are open, or one pulse at a time until the desired dose level is reached.

### Various Notes & Examples

Each of these non-limiting examples can stand on its own, or can be combined in various permutations or combinations with one or more of the other examples.

Example 1 can include a system for measuring chemical components. The system can include a dosing cylinder configured to receive and measure a fluid containing one or more chemical components, the dosing cylinder including a fluid sensor, a pump operably coupled to the dosing cylinder, the pump configured to drive the fluid to the dosing cylinder, and a controller in communication with the dosing cylinder and the pump. The controller can be configured to generate a signal to initiate driving of the fluid into the dosing cylinder, receive a signal from the fluid sensor in response to the fluid reaching a predetermined volume in the dosing cylinder, generate a signal to stop pumping of the fluid into the dosing cylinder, and dispersing the fluid from the dosing cylinder to a sterilization system in a dose of the predetermined volume.

Example 2 can include Examples 1, wherein dispersing the fluid from the dosing cylinder to the sterilization system is in response to an indication of a desired amount of sterilant filling the dosing cylinder.

Example 3 can include any of Examples 1-2, further comprising a second pump operably coupled to the dosing cylinder, the second pump for driving the fluid from the dosing cylinder to the sterilization system for a sterilization cycle.

Example 4 can include any of Examples 1-3, further comprising a fluid source coupled to the pump.

Example 5 can include any of Examples 1-4, further comprising a valve between the fluid source and the dosing cylinder, the valve for regulating flow of the fluid from the fluid source to the dosing cylinder, wherein the valve is operably coupled to the pump.

Example 6 can include any of Examples 1-5, further comprising a sterilization system fluidly coupled to the dosing cylinder, wherein the sterilization system is configured to receive the predetermined volume of fluid for a sterilization cycle.

Example 7 can include any of Examples 1-6, further comprising a valve between the dosing cylinder and the sterilization system for regulating flow of the fluid between the dosing cylinder and the sterilization system.

Example 8 can include any of Examples 1-7, further comprising a second pump between the dosing cylinder and the sterilization system, the second pump for driving the fluid into the sterilization system from the dosing cylinder.

Example 9 can include any of Examples 1-8, wherein the fluid sensor comprises an ultrasonic sensor configured to detect fluid height.

Example 10 can include any of Examples 1-9, wherein the controller is further configured to convert the detected fluid height to a fluid volume.

Example 11 can include any of Examples 1-10, further comprising a user interface device coupled to and in communication with the controller.

Example 12 can include any of Examples 1-11, wherein the controller is further configured to initiate driving of the fluid into the dosing cylinder direct driving of the fluid in a pulsatile fashion.

Example 13 can include any of Examples 1-12, wherein the controller is further configured to initiate driving of the fluid into the dosing cylinder direct driving of the fluid in a continuous fashion.

Example 14 can include a sterilization system including a sterilization module, a dosing cylinder coupled to the sterilization module, the dosing cylinder for receiving and measuring a fluid containing one or more chemical components, the dosing cylinder including a fluid sensor, a pump operably coupled to the dosing cylinder, the pump for driving the fluid to the dosing cylinder. and a controller in communication with the dosing cylinder and the pump. The controller can include a processor and a memory with instructions which, when executed, cause the processor to: initiate driving of the fluid into the dosing cylinder, identify a condition, based on data received from the fluid sensor, the condition indicating that the fluid has reached a predetermined volume in the dosing cylinder, stop pumping of the fluid into the dosing cylinder in response to the condition, and initiate driving of the fluid of the predetermined volume from the dosing cylinder to the sterilization module.

Example 15 can include Example 14, wherein the sterilization module comprises an atomizer configured to receive and atomize the fluid from the dosing cylinder.

Example 16 can include any of Examples 14-15, further comprising a second pump configured to drive the fluid from the dosing cylinder to the sterilization module.

Example 17 can include a method including the steps of controlling flow of a sterilant fluid into a dosing cylinder, measuring an amount of the sterilant fluid in the dosing cylinder, and stopping driving of the sterilant fluid into the dosing cylinder when a threshold amount is reached.

Example 18 can include Example 17, wherein controlling flow of the sterilant fluid comprises pumping the sterilant fluid in a pulsatile manner into the dosing cylinder.

Example 19 can include any of Examples 17-18, wherein pumping the sterilant fluid in a pulsatile manner comprises pumping a plurality of pulses of the sterilant fluid, wherein each of the plurality of pulses has a volume of about 5 μL to about 200 μL.

Example 20 can include any of Examples 17-19, further comprising driving the sterilant fluid from the dosing cylinder into a sterilization system for a use in a sterilization cycle.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system for measuring chemical components, the system comprising:
a dosing cylinder configured to receive and measure a fluid containing one or more chemical components, the dosing cylinder including a fluid sensor;
a pump operably coupled to the dosing cylinder, the pump configured to drive the fluid to the dosing cylinder; and
a controller in communication with the dosing cylinder and the pump, the controller configured to:
generate a signal to initiate driving of the fluid into the dosing cylinder,
receive a signal from the fluid sensor in response to the fluid reaching a predetermined volume in the dosing cylinder,
generate a signal to stop pumping of the fluid into the dosing cylinder, and
dispersing the fluid from the dosing cylinder to a sterilization system in a dose of the predetermined volume.

2. The system of claim 1, wherein dispersing the fluid from the dosing cylinder to the sterilization system is in response to an indication of a desired amount of sterilant filling the dosing cylinder.

3. The system of claim 2, further comprising a second pump operably coupled to the dosing cylinder, the second pump for driving the fluid from the dosing cylinder to the sterilization system for a sterilization cycle.

4. The system of claim 1, further comprising a fluid source coupled to the pump.

5. The system of claim 4, further comprising a valve between the fluid source and the dosing cylinder, the valve for regulating flow of the fluid from the fluid source to the dosing cylinder, wherein the valve is operably coupled to the pump.

6. The system of claim 1, further comprising a sterilization system fluidly coupled to the dosing cylinder, wherein the sterilization system is configured to receive the predetermined volume of fluid for a sterilization cycle.

7. The system of claim 6, further comprising a valve between the dosing cylinder and the sterilization system for regulating flow of the fluid between the dosing cylinder and the sterilization system.

8. The system of claim 6, further comprising a second pump between the dosing cylinder and the sterilization system, the second pump for driving the fluid into the sterilization system from the dosing cylinder.

9. The system of claim 1, wherein the fluid sensor comprises an ultrasonic sensor configured to detect fluid height.

10. The system of claim 9, wherein the controller is further configured to convert the detected fluid height to a fluid volume.

11. The system of claim 1, further comprising a user interface device coupled to and in communication with the controller.

12. The system of claim 1, wherein the controller is further configured to initiate driving of the fluid into the dosing cylinder direct driving of the fluid in a pulsatile fashion.

13. The system of claim 1, wherein the controller is further configured to initiate driving of the fluid into the dosing cylinder direct driving of the fluid in a continuous fashion.

14. A sterilization system comprising:
a sterilization module;
a dosing cylinder coupled to the sterilization module, the dosing cylinder for receiving and measuring a fluid containing one or more chemical components, the dosing cylinder including a fluid sensor;
a pump operably coupled to the dosing cylinder, the pump for driving the fluid to the dosing cylinder; and
a controller in communication with the dosing cylinder and the pump, the controller including a processor and a memory with instructions which, when executed, cause the processor to:
initiate driving of the fluid into the dosing cylinder,
identify a condition, based on data received from the fluid sensor, the condition indicating that the fluid has reached a predetermined volume in the dosing cylinder, stop pumping of the fluid into the dosing cylinder in response to the condition, and
initiate driving of the fluid of the predetermined volume from the dosing cylinder to the sterilization module.

15. The system of claim 14, wherein the sterilization module comprises an atomizer configured to receive and atomize the fluid from the dosing cylinder.

16. The system of claim 14, further comprising a second pump configured to drive the fluid from the dosing cylinder to the sterilization module.

17. A method comprising:
controlling flow of a sterilant fluid into a dosing cylinder;
measuring an amount of the sterilant fluid in the dosing cylinder; and
stopping driving of the sterilant fluid into the dosing cylinder when a threshold amount is reached.

18. The method of claim 17, wherein controlling flow of the sterilant fluid comprises pumping the sterilant fluid in a pulsatile manner into the dosing cylinder.

19. The method of claim 18, wherein pumping the sterilant fluid in a pulsatile manner comprises pumping a plurality of pulses of the sterilant fluid, wherein each of the plurality of pulses has a volume of about 5 μL to about 200 μL.

20. The method of claim 17, further comprising driving the sterilant fluid from the dosing cylinder into a sterilization system for a use in a sterilization cycle.
